(19) United States
(12) Patent Application Publication
(10) Pub. No.: US 2020/0368474 A1
(43) Pub. Date: Nov. 26, 2020

(54) TIP ADJUSTABLE STYLET

(71) Applicant: Yang Sun, San Francisco, CA (US)

(72) Inventor: Yang Sun, San Francisco, CA (US)

(21) Appl. No.: 16/416,386

(22) Filed: May 20, 2019

(51) Int. Cl. A61M 16/04

(52) U.S. Cl. CPC A61M 16/0488

(57) ABSTRACT

A flexible stylet to be inserted into and pushed out an endotracheal tube's for tracheal intubation under a visualization of a traditional or video laryngoscope. The stylet includes a rod which can be divided into a proximal segment, a distal segment which includes a bendable segment, and a tip segment. A retracting string is configured as a coil, spring, or wave-like shaped elongated flexible filament-like. Its proximal end is configured with a docking device which fastens to an endotracheal tube's proximal opening. The retracting string's distal end is coupled to the rod distal segment. The retracting string can generate pulling force when being stretched and elongated and therefore pulls back the rod distal segment back to form curves when the bendable segment is out of the ET distal aperture. A second string has two ends, one couples to the distal segment and the other to retracting string respectively.

### BACKGROUND

Tracheal intubation have been utilized in a wide variety of medical specialties in past decades for standard airway management. Many intubation tools have been developed to effectively insert an endotracheal tube into a patient's trachea through the vocal cords. Traditional metal direct laryngoscopies have been used for many decades which can provide a direct view of the vocal cords majority of the time but not in many difficulty airway situations. The currently commercially available video laryngoscopes can provide a better visualization if use properly. However, a good visualization is not equal to good intubation. Endotracheal tubes, brief as ET, have been utilized in the tracheal intubation.

An intubation process can often be difficulty because of the contours and obstacles encountered in a patient's airway. Patients' airway anatomy varies from patient to patient. Endotracheal tubes are generally made with a standard curvature to try accommodate and navigate these curves. However it is often necessary to change the preformed standard curvature to impart a greater bend to the tip of the tube in order to be inserted into the vocal cord opening under the vision of the direct laryngoscopy and video laryngoscope.

A number of currently available stylets have been developed to try to resolve these problems. One of the example is a metal malleable stylet which can be inserted into an endotracheal tube and be bent at a desirable curvature by an operator before starting intubation process, but the operator will not be able to change the curvature of the stylet as needed during intubation. Another stylet example is a rigid non-bendable metal stylet. The limitations of the rigid stylet in advancing the ET through to the vocal cords and into the trachea have been well documented.

In the recent decade, more and more commercially available Video laryngoscopes have created the need for more dynamically adjustable stylets to accommodate the unique angles and turns of a glottic opening area under vision of the video laryngoscope. An example of a commercially available articulating stylet is by Parker Medical Inc., U.S. Pat. No. 8,695,590 B2, which inserts into an ET and its tip can be manually adjusted when the ET approaches the glottis opening. Like all other types of stylets in which the distal tip of the stylet stays inside of an ET and positioned at distal opening of the ET. The stylet will make tip of the ET more rigid and often times make the curved tip of the ET very hard to align with a patient's glottic opening. As result, the ET's distal opening bevel often becomes stuck at the arytenoids, or anterior edge of the glottic opening. Thus, there is an increased chance of trauma to vocal cords and surrounding tissues. Such as recently commercially available “Truflex” stylet uses metal stylet to adjust the curvature of the distal tip of an endotracheal tube which can be quite traumatic to the airway anatomy. In all these prior arts, the tips of the stylet are positioned inside the distal opening of the endotracheal tube during the bending process which makes the bendable tip of the stylet hard manipulate and make the ET distal end become a rigid curve. Even though an operator can have a good view of the vocal cords by video laryngoscope, the stylet is difficulty to align with glottic opening and therefore difficult to guide the ET through the vocal cords opening.

### SUMMARY OF INVENTION

The present invention is a bendable and flexible stylet. The first aspect of the present invention is directed toward an intubation stylet for intubating a trachea. For the purpose of an endotracheal intubation, a traditional laryngoscope or a video laryngoscope is needed for visualization. Second, in many other clinical scenarios, often time a medical practitioner needs to reach or examine a patient's and animal's narrow body space or detoured passages which could not be normally reached by a human hand or straight shaped equipment. Those examination or treatment procedures may need a beddable stylet with a bending capability to reach these destinations. And the concept and basic design principle of the present invention is capable to be used to design similar equipment to be used in different medical fields even in an animal medical care.

When used in those clinical scenarios, a tubular structure can be used to replace an ET and is still able to be bent on its distal portion of the device by an operator under varies kind of visualizations directly or indirectly. In following descriptions, using the term of ET as an example, but an ET and a tubular structure can be used interchangeably.

The Present invention of a stylet will be described as three embodiments based on same inventive principle and concept. All three embodiments comprise an elongated flexible rod and a retracting string. Embodiment one and two also comprise a second string.

The rod, which can be formed as either a single piece or the joining of multiple sections, can be divided into a rod proximal segment and a rod distal segment. The length of the proximal and distal rod segment can vary in length. The rod distal segment can be further divided into a bendable segment and a tip segment. At the proximal end of the proximal segment, a rod ring is configured for an operator' finger to move the rod distally or proximally. The rod distal segment is to be pushed by operator's finger out of the ET distal aperture and thereby forms curvatures in the rod distal segment.

The retracting string is configured as a coiled-like, or spring-like or wave-like shaped elongated flexible filament-like or a thin belt-like. The retracting string's proximal end is configured to have a docking device which is removable and can fasten the proximal end of the retracting string to an edge of an ET proximal opening. The retracting string's distal end is coupled to the rod distal segment. The retracting string is a configuration of the coiled-like or spring-like or wave-like extensible and compressible structure that enables the retracting string to be extensible and elongated for a measurable distance. The final length of the retracting string after fully stretched can be pre-configured. The retracting string is configured to generate pulling force when being stretched and elongated and therefore pulls back the rod distal segment back to form curves in the rod when the rod is being pushed forward out of an ET.

First embodiment and second embodiment of present invention includes a second string with its two ends coupled to the bendable segment and to the retracting string respectively. A coupling point of the second string with the retracting string divides the retracting string into a string proximal filament and a string distal filament. The second string will impart a pulling force on the rod's bendable segment at a later stage of the curvature formation which occurs as the device is pushed out an ET or other tubular structures.

The device is configured to be inserted and pass partially through an ET lumen with distal portion of the device outside of the an ET distal aperture. In following description, an ET will be used to illustrate the principle and design of the present invention. However, the device can be used with other tubular structures for procedures other than intubation.

In all three embodiments of the present invention, a portion of the rod distal segment can be more or less positioned outside of the ET distal aperture before the device inserted into a patient's or animal's throat. An operator's dominate hand can hold proximal end of an ET and the docking device of the retracting string and one finger can put into the rod ring and control movement of the rod distal segment. Flexibility and resiliency of the rod distal segment and extensibility of the retracting string enable the operator to manipulate a distally located the bendable segment and the tip segment to form curvatures in the rod distal segment and thereby position or change a position of the distal tip of the device while the majority of the device is still inside the ET. Therefore an operator can align the tip of the device with the vocal cords opening and push the tip of the stylet device into and through the vocal cords opening. In contrast with prior arts, the distal portion of the present stylet is to be pushed further out of an ET's distal aperture and is capable of being manipulated by an operator to bend or form curves in the rod distal segment that is outside of the ET. This feature gives the present invention a great advantage to overcome above mentioned insufficiencies in the prior arts. The ET serves as a restraint means to keep the rod and the retracting string in proximity. And the upper edge of the ET's distal aperture provides an anchor point or a sliding point for moving the retracting the device back and forth.

The bending process, which occurs while pushing the stylet device through the ET's distal aperture, is a continuous process, but in present invention this process can be subjectively divided into three stages, eg. three staged pushes to describe curvature formation process and tip movement process. However an operator can choose to continuously push the rod without stopping at each stage until the tip segment of the rod enters the vocal cords opening.

The rod is a bendable member and a guiding member where its distal tip is advanced into vocal cords opening which guides the ET into the vocal cords opening and trachea. An operator is able to move the distal tip in front of and through the vocal cords by pushing or pulling the rod ring at proximal end of the rod. Once the tip is through the vocal cords, the ET can be slid along the stylet and into the trachea.

The materials forming the bendable rod, the retraction string and second string are preferably plastics, silicone, but can be materials such as other polymers, or metal or combination of all with properties of flexible, resilient and elastic. Preferably, the rod proximal segment is more rigid than the rod distal segment and the rod distal segment is more rigid than the retracting string.

An operator's finger can control a pushing force on the rod's proximal end to form different curvatures along the bendable segment, and thereby move the rod tip segment according to each patient's specific airway anatomy under the vision of a laryngoscope. Proximal sliding or pulling of the rod ring decreases the curvature of the bendable segment and the distal movement of the rod ring increases the curvatures of the bendable segment.

### BRIEF DESCRIPTION OF THE DRAWINGS

FIG. 1 —A left side view of the first embodiment of the present invention before insertion into a tubular structure, the retracting string is in a loose state, and the string hook is not in contact with the rod. The rod bendable segment is in preformed curved shape FIG. 2 —A left side closed-up view of distal portion of the device of the FIG. 1 with the second string designed alternatively as a coiled like extensible and compressible structure.

FIG. 3—Left side view of the rod being inserted into a tubular structure or an ET, to demonstrate flexibility and elasticity of the tubular structure curvature and the rod, and their interacting when the rod is in the set-up state. Dotted line shows the curvature and shape of the tubular structure before the rod is inserted into the tubular structure. If a tubular structure is an endotracheal tube, the rod proximal segment is constructed more rigid than the ET. In order to clearly demonstrate features of the rod the restricting string and the second string are not shown.

FIG. 4a-An alternative design of a semi-plug like docking device for the retracting string.

FIG. 4b-An alternative design of a crescent shaped plate as a docking device for the retracting string.

FIG. 5a-5d-Schematic drawing of three stages of curvature formation at the rod distal segment which is generated by an operator's first, second, and third push in the first embodiment of the present invention.

FIG. 5a-Left side view, the device inserted into an ET and in a set-up position. A portion of the rod distal segment, the retracting string and second retracting string are positioned outside of an ET distal aperture 48.

FIG. 5b—Left side view, the device has had the first stage of the curvature formation after operator's first push, the rod distal segment 12, the retracting string, and the second string 17 are further pushed out of the ET distal aperture.

FIG. 5c-Left side view, the second stage of the curvature formation after second pushes.

FIG. 5d-Left side view, the third stage of the rod's curvature formation after operator's third push, the distal portion of the rod, second string and distal portion of the retracting string are outside of the ET distal aperture. Dashed lines represent that if the string distal filament or the first bending section is against upper pharyngeal wall, the tip segment and the first bending section can be pressed down.

Please note, in all FIG. 5a, 5b, 5c, 5d, an ET is imaginarily held in the same position when the first, second and third push are executed. The second string is gradually moved from a more horizontal position at the set-up configuration to reverse its orientation. At third stage the second string becomes a part of the retracting means to hold back the bendable segment of the rod and thereby making the first bendable junction 28 bend.

FIG. 6a-A closed-up view from top, showing the bendable side connection 31 the second embodiment of the present invention and other structures of the distal portion of the device.

FIG. 6b-A closed-up view from top, showing an alternative design of the bendable side connection 31 in the second embodiment of the present invention FIG. 7a,b,c,d—A second embodiment of the present invention.

FIG. 7a-Left side view of the second embodiment, the device is inserted into an ET and in a set-up position. The tip segment 39, a portion of the rod bendable segment 30, a portion of the retracting string and second string 37 are positioned outside of an ET distal aperture.

FIG. 7b-Left side view of the second embodiment with first stage of the rod's curvature formation after operator's first push, the bendable segment of the rod, the retracting string, and the second string are further pushed out of the ET distal aperture.

FIG. 7c-Left side view of the second embodiment in second stage of the rod's curvature formation after the operator's second push. The first bendable junction 28 has been pushed out of the ET distal aperture.

FIG. 7d-Left side view of the third stage of the curvature formation after operator's third push, in addition to changes in the previous stages, the second string is participating curvature formation and becomes a part of pulling means.

Please note that during above three pushes, the second string 37 is gradually be pulled to a more horizontal position and at third stage where it becomes part of the retracting means to pull up the tip segment distal end 33 and thereby making the tip distal curved region38 point down.

FIG. 8—A left side view of the third embodiment of the present invention in a relaxed state, before insertion into a tubular structure. The rod bendable segment 55 is a preformed bow shape.

FIG. 9a-Left side view of the third embodiment with the device inserted into an ET and in a set-up position.

FIG. 9b—Left side view of the third embodiment at the first stage of the rod's curvature formation after operator's first push, the more bendable segment of the rod and the retracting string are further pushed out of the ET distal aperture.

FIG. 9c—Left side view of the third embodiment at the second stage of the rod's curvature formation after operator's second push. The whole bendable segment of the rod is pushed out of the ET distal aperture. The first bendable junction 28 is at just outside of the ET distal aperture and will be bent up.

FIG. 9d-Left side view of the third embodiment at the third stage of the curvature formation after operator's third push, at this stage the tip segment is being pulled down via the bendable junction section and, the tip distal section is pointing downward.

FIG. 10a-Left side view of the third embodiment used in different way by an operator in a set-up position. A length of the string proximal filament 22 is placed outside of the ET proximal aperture. When an operator pushes the rod distally, the portion of the retracting string outside of the ET's proximal aperture will slide into the ET lumen and the string hook 21 will engage on the anterior edge of the ET proximal aperture.

FIG. 10b—Left side zoom-up view of a distal portion of the FIG. 10a, with the string distal attachment designed as a string attachment plate 53b to couple to the tip middle section 57.

### DETAILED DESCRIPTION OF THE PREFERRED EMBODIMENT OF THE PRESENT INVENTION

The following description will use an ET during an airway management as an example to illustrate the device's parts and basic design concept.

A total of three embodiments based on the same novel concept and design are described. Though, more alternative designs can be derived from the disclosed preferred embodiments. And elements of each embodiment may be replaced by other elements of other embedments. They are all within the scope of the protection of the present invention.

As used in this application, the word “member” refers to a structural element. For purpose of the drawing and description, the use the word of “proximal” refers to an end of the stylet device closest to the operator when an operator holds the device and is ready to insert the device into a patient's mouth. The reference to the term “distal” refers to an end that first enters into a patient's mouth and is opposite end of the proximal end. Therefore, the distal tip of the device is always points distally. Therefore, an operator push of the bendable member distally, it can also be described as a push “forward or down”. The term ET represents an endotracheal tube or a tubular structure in general. The word “posterior” refers toward a back side of an operator or patient's body or patient's back pharyngeal wall. The word “anterior” refers to the front side of an operator or patient body or toward the glottic opening. A commonly used ET is longitudinal hollow tube with a proximal aperture and a distal aperture. The proximal aperture has an adaptor for connecting with a respiratory machine. The adaptor will be removed before the device is inserted into an ET. During intubation, the ET has always been held in an orientation where the ET distal aperture bevel is facing to an operator's left. Therefore, for purpose of easy description and understanding, all drawings are views from left side, therefore, the ET distal aperture bevel is always facing toward the reader. A concave side of an ET will be described as “anterior aspect or anterior wall” because the ET's anterior wall is always inserted toward front side of patient's body. Vice versa, a convex side of the ET will be described as the “posterior aspect or posterior wall”. Since this device can be used in a patient or an animal, the term of “patient” represents human patient and animals.

### First Embodiment

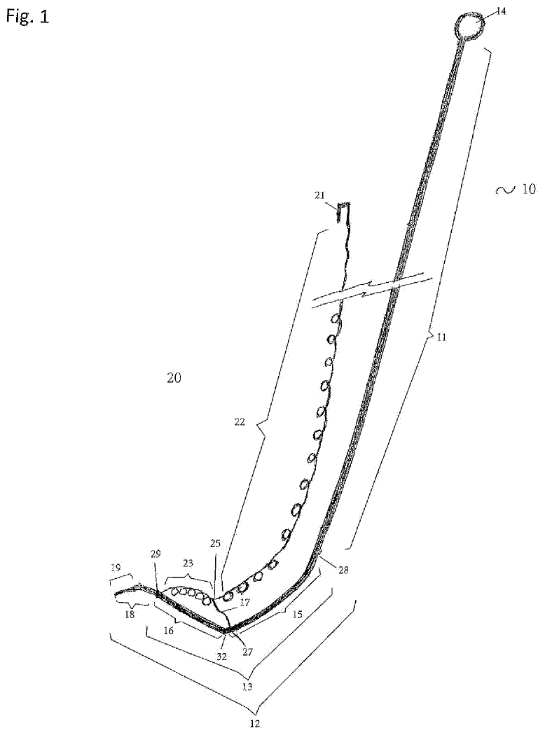

FIGS. 1 and 2, showing in the first embodiment of the present invention, the device is in relaxed and loosed state before inserted into an ET. The device includes a rod 10, a retracting string 20 and a second string 17.

The rod is a flexible member. From the proximal to distal, the rod 10 includes a rod proximal segment 11, a rod distal segment 12. The rod distal segment 12 can be further divided into a bendable segment 13, and a tip segment 18. A circle shaped ring, call rod ring 14 is coupled to the most proximal end of the rod 10. From the proximal to distal direction, the bendable segment 13 can be further divided into first bending section 16, and second bending section 15. The rod proximal segment has a proximal end and a distal end. The distal end of the rod proximal segment 11 couples with proximal end of the bendable segment 13 to form a 120-170 angle at the junction, called first bendable junction 28. The junction between the first bending section 16 and second bending section 15 is configured with a 100-160 degree angle, called third bendable junction 32. Proximal end of the tip segment 18 couples the distal end of the second bending section 15 and is configured with a 100-160 degree angle, called second bendable junction 29. The second bendable junction 29 is designed to have the tip segment 18 to be bent downward when the tip segment 18 meets a resistance or pressure from above, such as against the anterior pharyngeal wall during operation. The third bendable junction 32 can be preformed with an upward bend which will bend more when the second bending section 15 is pulled by the retracting string 20 at an early curvature formation. The first bendable junction 28 is design to be bent when the second bending section 15 is pulled by the second string 17 and when the first bendable junction 28 is pushed outside of the ET distal aperture.

The first bending section 16 and second bending section 15 are resiliently flexible and can bend when an operator is pushing the rod ring 14 distally and the retracting string 20 is pulling on the first bending section 16. The third bendable junction 32 is configured to readily bend up and plays a major role in curvature formation before the first bendable junction 28 starts to significantly bend. The first bendable junction 28 can be only bent any significate amount when the first bendable junction 28 has been pushed out the distal opening of the ET 48. The first bendable junction 28 is configures to withstand the pushing force without being significantly bent and can transmit the pushing force from the rod proximal segment 11 to the bendable segment 13 when it is inside of the ET's lumen.

Alternatively the bendable segment 13 can be configured as more than two bending sections with more than one bendable junction in which each bendable segment and bending section junction can contribute a smaller amount of total amount of bend.

Different segments of the rod are designed to have different cross section diameter which cause different stiffness to withstand different physical forces by using same materials during manufacture. Or alternatively, the different segments of the rod can use different materials with different stiffness. The rod proximal segment 11 has a bigger diameter than the rod distal segment 12. The bigger diameter make the rod proximal segment 11 is less flexible and strong enough not to be significantly bent and mostly keep its original shape when being pushed by an operator, therefore to transmit an operator's pushing force distally to the bendable segment 13. The smaller diameter of the bendable segment 13 and the tip segment 18 make they easily to be bent. The shape of cross section of the rod 10 can be same or changeable over its length. The cross section can be many geometric shapes, such as square, rectangle, trapezoid, triangle, round, semi-circle, ellipse, semi-ellipse, diamonds, or combinations. Alternatively the bendable segment 13 and the tip segment 18 can be made with different materials with more flexibility but with same diameter as the rod proximal segment 11. The bendable segment 13 is a portion of the rod 10 that forms curves and thereby moves the tip segment 18 to a target position.

The tip segment 18 is a bent curve-shape with its tip curved region 19 slightly bending down. The tip curved region 19 is about ¼ to ¾ of whole length of the tip segment 18 and is also called a tip of the rod or a tip of the device. Alternatively the tip segment 18 also can be configured as crescent shape. At the second bendable junction 29, the diameter of the tip segment 18 becomes smaller and continues to tapper smaller until the distal end of the tip segment 18. Therefore, the tip curved region can be easily pressed and bent down in addition to the bendability of the second bendable junction 29. And the second bendable junction 29 is also configured to be easily bent down if the tip segment 18 meets a resistance from above. The retracting string distal end is coupled to or just proximal to the distal end of the first bending section 16, in other word to just proximal to the second bendable junction 29. Therefore the retracting string 20 will not directly exert a pulling force on the second bendable junction during curvature formation.

However an operator can intentionally push the tip segment 18 against the upper pharyngeal wall to create a resistance or pressure from above therefor to bend the second bendable junction 29. But the resilient property of the tip segment 18 will resume its original shape once the resistance is passed during an operator's pushing the rod 10. Therefore the tip segment 18 can “navigate” within a patient's pharyngeal space and take a little “detour” then into the vocal cords opening if the vocal cords are poorly visible or even only can be estimated. Once the tip distal segment has entered the vocal cords opening, the operator' hand will feel “loss of resistance” because the vocal cords opening has no resistance, then the ET can be slid along the device into the trachea to finish intubation.

The surface of the tip segment 18 is very slippery either by well lubricating before its use or being made with a low coefficient of friction materials, such as coating of polytetrafluoroethylene (PTFE) material or similar materials, or combination the both.

The retracting string 20 is a continuous elongated flexible extensible string or a belt and can be configured as three different shaped longitudinally: wave-like, coiled-like or spring-like. The coil-like or spring-like or wave-like structure can be configured as a portion of the retracting string or whole length of the retracting string 20. A cross section area of the retracting string 20 is smaller than the rod 10. It is designed to be much more flexible than the bendable segment 13 and same time can hold back the bendable segment 13. It is a continuous string 20 but can be divided or described as a proximal filament 22 and a distal retracting filament 23. The retracting string's distal end is attached to the distal end of the first bending section 16 just proximal to the second bendable junction 29. The retracting string's proximal end become thicker and wider to be configured as a hook, called string hook 21, to hook on the anterior edge 41 or lateral edge of the ET proximal aperture 42. Because the string hook 21 cannot be moved distally with the retracting string during the rod distal movement, the string hook 21 will hold the retracting string 20 proximal end at its original place and let the coiled-like or spring-like or wave-like extensible structure be extended and elongated. Therefore, when the rod is pushed distally, the tension will be built on the retracting string 20 and the coiled-like or spring-like or wave-like structure will generate a counterforce by tis elastic recoiling. This counterforce is a force to pull up and bend the bendable segment 13 during curvature formation. Final length of the retracting string 20 after being completely stretched can be determined during manufacture therefore to have a pre-measured extensibility. Degree of extensibility can be altered by the number of each coiled-like or spring-like or wave-like structure along its length. The string proximal filament 22 and the string distal filament 23 can have same or different configuration of the extensibility. Extensibility of the retracting string 20 is essential for the bendable segment 13 moving distally out of the ET distal aperture 48. The string distal filament 23 is configured to not be fully extended with residual extensibility at late stage of the curvature formation. However, the string proximal filament 22 may or may not be fully extended to its final length. The string proximal filament 22 can be configured to have a portion or whole length as a coiled-like or spring-like or wave-like structure.

Referring to FIG. 1, 5a, 5b, 5c, 5d, a short length of a wave shaped string or a filament or belt-shape segment, called second string 17, is configured. Alternatively the second string 17 can be configured as coiled or spring shaped structure, FIG. 2. The second string 17 has two ends or attachments. The first end, called second string's first end 25, is coupled to a dividing point of the string distal filament 23 and string proximal filament 22. And the second end is coupled to near distal end of the second bending section 15 where is just proximal to the third bendable junction 32, called second string' second end 27. Alternatively the second string's second end 27 can be configured to couple to the third bendable junction 32. At set-up position the second string 17 is relaxed and is slanted between the retracting string 20 and the bendable segment 13 with the second string' first end 25 more distally positioned than the second string' second end. From beginning of the push to the last stage push, the second string 17 is gradually stretched and its two ends will reverse their orientation with the second string' first end 25 becoming more proximal than the second string' second end 27. Alternatively the second string 17 can be configured as a straight line.

The function the second string 17 is to be used as part of the pulling means to pull the second bending section 15 during later stage pushing. During last stage of pushing, the second string 17 is fully taut and the second string 17 become a straight line or near a straight line with the string proximal filament 22 to pull the second bending section 15 backward by using the first bendable junction 28 as a bending point. At same time the string proximal filament 22 is fully or near fully stretched and together with the second string 17 to pull the second bending section 15 up. At this moment, the tension on the string proximal filament 22 and on the second string 17 is high and tension on the string distal filament 23 is lower.

The whole retracting string 20 will be participate the pulling the first bending section up before the second string 17 is pulled and become the part of pulling means. When the second string 17 is pulled straight and consequently pulls the second bending section 15 up, the string distal filament 23 is still in wave-shaped and still has some extensibility which will allow the second bending section 16 to be pressed down by reversing some previously bending by the third bendable junction 32.

Alternatively, the retracting string 20 can be configured as a straight filament and just simply be folded or loosely lie inside of the ET lumen at set-up stage. When the rod bendable segment 13 is pushed out of the distal opening of the ET 48, the retracting string 20 will be pulled out of the ET distal aperture and then to pull the bendable segment 13 back to form curvatures.

The rod proximal segment 11 and rod bendable segment 13 have a larger cross section area than the retracting string 20 and therefore are stiffer and stronger than the retracting string 20. Alternatively, the retracting string 20 can be made with different materials with more flexibility and elasticity. When an operator push the rod ring 14 and makes the rod 10 a distal movement, the rod 10 will pull the retracting string 20 distally. The retracting string 20 will be gradually tighten, elongated and gradually increase its tension along the retracting string 20 and generate a pulling force due to its elastic recoil property. The retracting string 20 will hold the bendable segment 13 backward to bend and form a curve which will move up the tip segment 18 position.

This retracting force and the rod's distal movement create the bending. The bendability and length of the rod bendable segment 13, flexibility and the number of the bendable junctions and the bending sections, and extensibility of the retracting string 20 and total length of the retracting string 20 in comparison with total length of the rod 10 are essential for curvature formation outside of the ET distal aperture. By manipulating those variables, the present invention can be modified into different embodiments and obtain an optimal result. They all are within protection of the present invention.

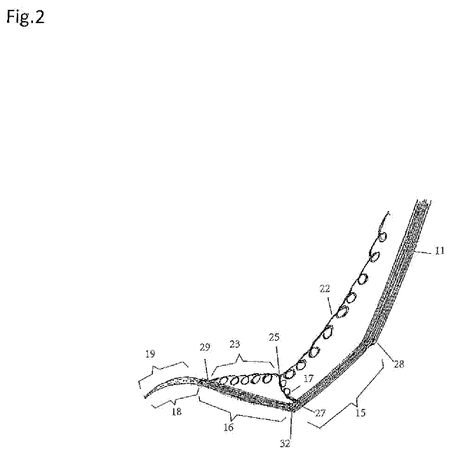

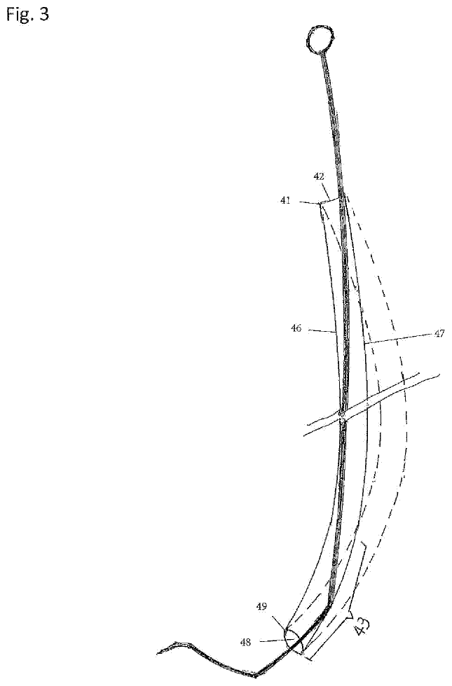

FIG. 3 is showing a commonly used ET and interact between the ET preformed curvature and the rod's flexibility and rigidity. The ET has proximal aperture 42 and a left faced beveled distal aperture 48. The ET distal aperture 48 has a upper edge 49. The ET has an anterior wall 46 and posterior wall 47. The ET has performed rainbow like curve which is changeable. From the ET distal aperture toward the proximal direction, about bout 3-7 cm centimeter length of the distal portion of the ET is called ET distal segment 43. The rod proximal segment is configured with more rigidity the bendable segment. The proximal segment of the rod is configured as a straight or almost straight and will reduce the curvature of the ET after inserted in the ET lumen. The bendable segment's rigidity is slightly more than the ET's rigidity in the first and the second embodiment can make the ET distal segment 43 bend up more in addition to the ET original curvature, at same time the ET distal segment 43 can reduce some of the preform curvature of the bendable segment. But in the third embodiment, the ET distal segment 43 cannot reduce of the curvature of the bendable segment 55. During process of the bending, the upper edge 49 of the ET distal aperture 48 can serve as an anchor point or sliding point for the retracting string 20 to slide back and forth when the rod 10 is pushed or pulled along the ET lumen by an operator.

And as above described, at beginning of set up position, the distal portion of the device is outside of an ET distal aperture. However alternatively, the whole device can put inside of the ET at the beginning and slide out during the operation. In both scenarios the curvature formation processes is all executed outside of the ET distal aperture 48. This is important feature of the present invention.

Alternatively the retracting string 20 can be designed to be pulled by an operator causing the retracting string 20 to pull the rod bendable segment 13 and form the curves. In a further alternative embodiment the retracting string 20 can be designed as a retracting string pulled proximally by an operator in addition to the rod 10 being pushing distally. These variations of the design are using same principle and concept the present invention and are within protection or the present invention.

Alternatively the tip segment 18 can have a same diameter in cross section with the second bending section 16 which will give the tip segment more rigidity to withstand some resistance. The second bendable junction 29 will be a bending point for the tip segment binding down.

In other alternative embodiments, the tubular structure can be replaced by other structures with similar shape, such as fenestrated tubular structure, or not similar a tubular structures but having same function as a tubular structure to hold the rod and retracting string in proximity, such as a coiled belt wrapping around the rod and the retracting string intermittently or continuously along the retracting string from proximal end to distal portion. And further, the present invention can embed a bundle of fiber-optic fiber into the rod along its long axis to make the device has its own “vision” which can make the device capable to be used independently without a laryngoscope. They are all within scope of protection of this invention.

FIG. 4a, show an alternative design of the docking device 24. The docking device 24 is configured as a semi-circled plug by horizontally folding of many times of the proximal end of string proximal filament 22, called docking plug 24. FIG. 4b show another alternative design in which the proximal end of the retracting string can be attached to lower surface of a crescent shaped plate, called docking plate 26. The both docking plug 24, docking plate 26 and string hook 21 all are docking device which is to reversibly engage the retracting string proximal end to the anterior or lateral edge of the ET proximal aperture 42. A semi-circled crescent shaped plate and semi plug have a bigger diameter than ET proximal opening; they will not being pulled into the ET lumen by the retracting sting 20 therefore to fasten the proximal end of the retracting string onto the ET proximal opening. Same time they will not obstruct movement of the rod.

The concept of the docking device includes the string hook 21, or alternatively the docking plug 24 and the docking plate 26. They all are made with bigger size than the retracting string and more rigidity than the retracting string 20 therefore no extensibility. They can be molded together with the retracting string 20 during the manufacture.

The curvature formation is a continuous process. An operator can choose to push the rod continuously or intermediately. In order to easily describe details of this continuous process, it is better to subjectively divide this continuous process into three stages of curvature formation or three stage of pushing: first push, second push, and third push. The operator can stop pushing the rod at any stage, once an operator's finger pushes the rod ring 14 and obtains a desirable position of the tip segment under a vision of a laryngoscope or video laryngoscope. And the operator can push the rod ring 14 and also pull the rod ring to align the tip of device with vocal cords opening. In addition, the operator can rotate or move the ET with the device back and forth to aim to vocal cords opening.

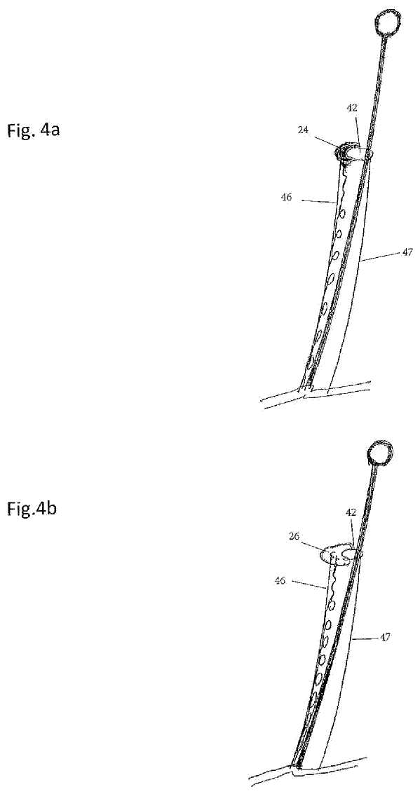

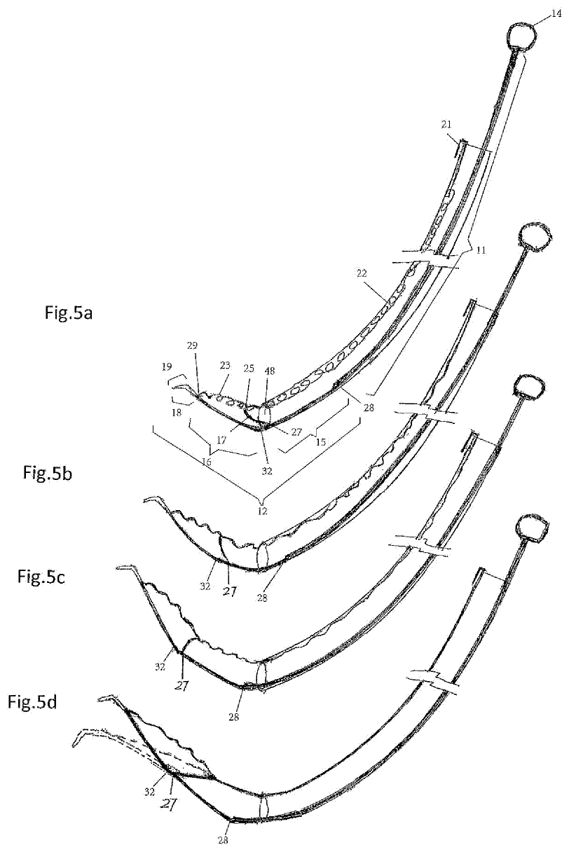

Referring to FIG. 5a, to prepare to use the device, an operator will lubricate bendable segment 13, most of the rod proximal segment 11, and most of the retracting string 20. FIG. 5a showing a left side view, the device has been inserted in to an ET 40 and the string hook 21 has been hooked on the anterior edge of the ET proximal aperture. It is called set-up position. And now an ET and the device together are ready to be inserted into patient's throat.

A length of the rod distal segment 12 and a correspondent length of the distal portion of the retracting string 20 are positioned outside of the ET distal aperture. A length outside of the ET distal aperture can be variable depending on each patient or animal, or operator's experience. Before inserting into a patient's mouth, the operator's one finger of a dominated hand, preferably index finger, will be placed into the rod ring 14 and other fingers will gripe a proximal portion of the ET.

In the set-up position, the retracting string 20 is no longer in a loose fashion. The coiled-like or spring-like or wave-like retracting string still remains un-pulled and un-extended coiled state. The second string 17 is in relaxed state and positioned between the retracting string 20 and the bendable segment 13.

Referring to FIG. 5b, the first push will slide more of the bendable segment 13 distally out of the ET distal aperture 48 which will pull retracting string 20 in same direction and stretch the retracting string about ⅓-¼ of its total extensible capability, also called first stage of the curvature formation. At same time, elastic recoiling force of the coiled-like or spring-like or wave-like structure of the retracting string 20 will pull the first bending section 16 back to cause the third bendable junction 32 to bend. The first bending section 16 and the second bending section 15 will also start to bend and participate the curvature formation. The second string 17 becomes more vertically positioned, but still has no tension. And the tip segment 18 is elevated.

Referring to FIG. 5c, the second push will slide the whole length of the bendable segment 13 out of the ET distal aperture 48 which will further pull retracting string 20 distally. After the second push, extensibility of the retracting string 20 will be used up about ⅔- 2/4. In addition to the first push, the natural elastic recoiled capability of the retracting string 20 is additively generating more pulling force to pull the first bending section 16 backward. At same time the second bending section and first bending section will be bent more, so does the third bendable junction 32. The first bendable junction 28 has been pushed out of the ET distal aperture 48. The first bendable junction 28 has started to bend. The second string 17 is becoming more vertically positioned and its two ends are reversing its proximal and distal orientation. The second string 17 is tightening and starting to pull the second bending section 15. And the tip segment 18 is elevated more.

Referring to FIG. 5d, the third push will push the first bendable junction 28 further out of the ET distal aperture, the retracting string proximal filament 22 has used up its extensibility 3/3-¾ and becomes straight or nearly straight. During third push, the second string 17 will become fully stretched and will become strait line or almost straight line with the string proximal filament 22 to pull the second bending section 15. The string proximal filament 22 and the second string 17 will pull up more the second bendable section 15 by bending the first bendable junction 28. At same time the third bendable junction 32 will bare less tension because the 17 now take part in pulling and make the string distal filament 23 have a less tension. Third push will generate a different shaped curvature which is largely produced by the first bendable junction 28. Because even after third push the string distal filament 23 has not completely used up its extensibility, if there is a resistance or pressure from above, the string distal filament 23 and the first bendable section 16 and the third bendable junction 32 can reverse some of the bending executed in previously first and second push. And if the tip curved region 19 encounters a pressure from above during this process, the tip curved region 19 and the second bendable junction 29 can be further bent down. Final goal is aligning and moving the rod tip 18 into the vocal cords opening. During the third push the tension on the string proximal filament 22 is high, but the tension on the string distal filament 23 is lower.

Due to flexibility and elasticity of the materials made of the bendable segment 13 and the string proximal filament 22, even after third push the operator is still able to push the rod more distally in relatively small amount to adjust the tip segment 18 position.

### Second Embodiment

In following description, if an element is configured same with same function as in the first embodiment, same name and number label will be assigned. If an element has same function but configuration is not same as in the first embodiment, then a same name but a different number label will be assigned.

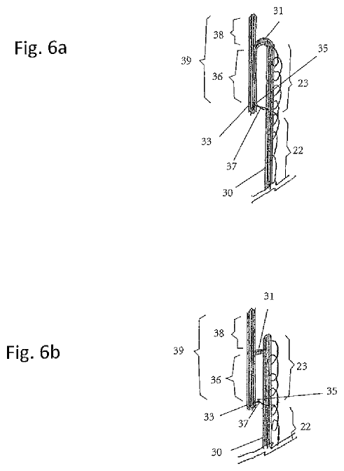

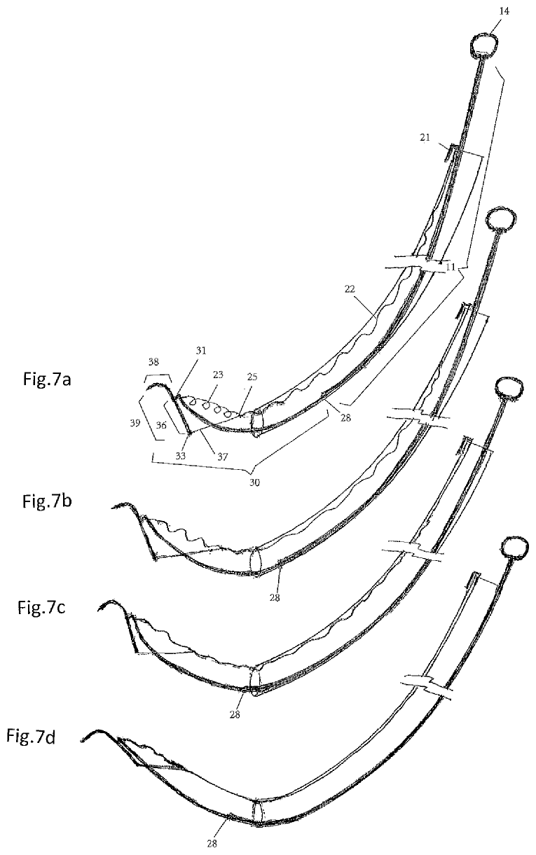

FIGS. 7a, 7b, 7c, 7d and 6a, 6b, based on the same concept and principle, the second embodiment also comprises three components, a rod 10, a retracting string 20 and a second string 37.

The coil-like or spring-like or wave-like extensible structure of the retracting string 20 and the string hook 21, the rod proximal segment 11 and the rod ring 14 are configured completely same as in the first embodiment.

However there are some differences. First, the bendable segment 30 is configured as a smooth preformed bow shaped segment without further dividing into the smaller sections. Second, the tip segment 39 couples with distal end of the bendable segment 30 with its right or left side by a short side connection, instead of end to end connection. Third, the tip segment proximal end is connected and pulled by a second end the second string 37.

From the proximal end to distal end, the rod 10 includes a rod proximal segment 11, and a bendable segment 30, and a tip segment 39. The tip segment is still to be advanced into vocal cords opening first same as the first embodiment.

The rod proximal segment 11 are constructed same as in first embodiment, and is almost straight but can be bendable under a strong force. The rod proximal segment 11 is made of materiel with more rigidity and can make the ET 40 more straight from its original curvature. The bendable segment 30 is configured as a smooth bow shaped curve without further dividing into smaller section units. The bendable segment 30 has more flexibility than the rod proximal segment 11. The bendable segment is more curved than the curvature of the ET distal segment 43 and rigidity is slight more than the ET. The original curvature of the ET distal segment can be bent more by bendable segment 30 in some extent and same time the original curvature of the bendable segment can be bent slightly less by the ET distal segment 43 when the bendable segment 30 is inserted into the ET lumen.

The junction of the rod proximal segment 11 and the bendable segment 30 is a bendable junction, same as in the first embodiment, also called first bendable junction 28. The first bendable junction 28 will be constructed with same stillness and bendability and working same way as in the first embodiment.

Referring FIG. 7a, the tip segment 39 is configured as an upside-down “J” shape with a curve on distal end of the tip segment 39, called the tip distal curved region 38. The rest length of the tip segment 39 is called tip proximal segment 36. The most proximal end of the tip segment 39 is called tip segment proximal end 33. The length of the tip distal curved region 38 is about ⅓ to ⅕ of total length of the tip segment 39. Alternatively the tip segment 39 can be configured as a crescent, sickle shaped. At middle area of its length and on the right or left side, the tip segment couples with the distal end of the bendable segment 30 via a short segment, called bendable side connection 31. The bendable side connection 31 is preferably configured as a the distal end of the bendable segment being bent and turned left or right to couple with right or left side of middle area of the tip segment long axis.

FIG. 6b is a zoomed-up view from the top, showing that preferably the bendable side connection 31 is formed when the distal end of the bendable segment 30 makes a smooth turn to couple to right or left side of the middle region of the tip segment 39. This smooth turn functions as a “joint” to connect the bendable segment 30 and the tip segment 39 and is to be bent or twisted under an external force. Alternatively, the bendable side connection 31 can be configured as a short segment or joint-like structure anywhere between the tip segment 39 and the bendable segment 30, FIG. 6b. The bendable side connection 31 can be made by same or different materials as the tip segment 39 and the bendable segment, preferably by same plastic materials or silicon or other type of polymers with similar properties. The bendable side connection 31 can bent or twisted under an external force. Therefore, the whole tip segment 39 can be moved like a lever with its tip distal curved region 38 moving down and the tip segment proximal end moving up, when the tip segment proximal end 33 is pulled up.

FIG. 7a, the second string 37 is preferably configured a straight filament or belt with two ends. Alternatively, it can be configured as curve or a wave or coiled shape. The second string 37 has two ends. The first end, also called the second string's first end 25 attaches to the dividing point of the string proximal filament 22 and the string distal filament 23. The second end of the second string, called second string distal end, is to attach to the tip segment proximal end 33. Therefore, the tip proximal segment 36 and the second string form a 70-140 degree angle in set-up position, called string pulling angle 35. When the tip segment proximal end 33 is pulled up by the second string, the whole tip segment 39 will be moved like a lever with the tip distal curved region 38 moving down. The string pulling angle 35 will become a more duller angle, or degree of the angle will become larger. At the final stage of the push, the string pulling angle 35 can even disappear when the tip proximal segment 36 and the second string become a straight line or near a straight line.

If the tip distal curved region 38 of the tip segment 39 encounters the resistance from above during curvature formation when the tip segment 39 is elevated by the bendable segment 30. The tip segment 39 can move like a lever and the tip distal curved region 38 will tilt down. But the resilient property of the tip segment 39 will resume its original shape once the resistance is passed. Therefore, even if the location of vocal cords can only be barely seen or can only be estimated, the operator can push the rod toward that direction to “look for” the vocal cords opening. Once the tip curved region 38 has entered the vocal cords opening, the operator will feel “lose of resistance” as in the first embodiment. This maneuver is same as in the first embodiment. The surface of the tip segment 39 is also very smooth and slippery.

Function of the second string 37 is to be used as a part of the pulling means to pull the tip segment proximal end 33. During the pushing, the second string gradually aligns with the string proximal filament 22 as straight line or an almost straight line and together with the string proximal filament 22 to pull the tip segment proximal end 33. The tip segment 39 is configured stiffer or stronger than the tip segment in first embodiment. This stiffness will make the tip segment 39 moving like a lever when the tip segment proximal end 33 is pulled up by the second string 37.

Distal sliding of the rod will stretch the retracting string 20 and make the bendable segment 30 be pulled back therefore to increases the bending of the bendable segment. The distal end of the bendable segment 30 will move up thereby moving whole the tip segment 39 in a higher position. In addition to the preformed bow curve shaped, the further bending of the bendable segment 30 is performed by the string proximal filament 22 and the string distal filament 23.

Same as in first embodiment, the rod proximal segment 11 has a larger cross section and are stiffer and not easily bendable than the bendable segment 30. When an operator push the rod ring and make the rod a distal movement, the rod will pull more the retracting string 20 outside of the ET distal aperture 48 and elongate the retracting string 20. Consequently the retracting string 20 will hold the bendable segment backward to form curves. These two opposite forces create the bending. The bendability of the rod bendable segment 30 and the extensibility and counterforce of the retracting string are essential for curvature formation.

Referring to FIG. 7a, at a set-up position, an ET's proximal end is held by an operator's hand same as in the first embodiment. The device has been inserted into an ET lumen and the tip segment 39 and some of the bendable segment 30 and the retracting string 20 are positioned outside the ET distal aperture 48. How much they are positioned outside of the ET distal aperture can be variable according of each operator and patient's situation. In set-up position, the string hook 21 is hooked on the edge of the ET proximal aperture 42. The retracting string's the coiled-like or spring-like or wave-like structure still remains in an un-extended state.

Referring to FIG. 7b, the first push will slide more of the bendable segment 30 distally out of the ET distal aperture 48 which will pull the retracting string 20 in same direction and stretch the coiled-like or spring-like or wave-like structure about ⅓ to ¼ of its total stretching capability or its final total length. At same time, the elastic recoiling force of the retracting string will pull the bendable segment 30 back to be bent up. Consequently the tip segment 39 is elevated. The second string 37 will begin to pull the tip segment proximal end 33. The string pulling angle 35 becomes more dull angle.

Referring to FIG. 7c, the second push will slide the whole length of bendable segment 30 out of the ET distal aperture 48 which will further pull the retracting string 20 distally. After the second push the retracting string will use ⅔ to 2/4 of its total extensibility or will have ⅓ to 2/4 to reach its final length. In addition to the first push, the natural elastic recoiled capability of the string proximal filament 22 is additively generating more pulling force to pull the second string 37. At same time the bendable segment becomes deeper bow shaped curve. The string proximal filament 22 pulls the second string 37, and consequently the second string 37 pull the tip segment proximal end 33 to up and in a proximal direction. The tip segment 39 is tilted by using the bendable side connection 31 as “bending point.” The tip distal curved region 38 tilts further down direction. The string pulling angle 35 becomes a more dull angle.

Referring to FIG. 7d, third push, the rod 10 is pushed and slid more distally, the first bendable junction 28 is out of the ET distal aperture and will be bent significantly. Since the retracting string 20 has used all or ¾ its extensibility and almost reached or near reached its final length. The retracting string 20 would be further pulling the distal end of the bendable segment 30. The second string 37 will align with the string proximal filament as a straight line or a near straight line and further pull up the tip segment proximal end 33. The tip distal curved region 38 tilts further down. The string distal filament 23 is stretched to its final length or near its final length.

In addition, the operator can intermittently push and release pressure on the rod ring 14 to align the tip distal curved region 38 with the vocal cords opening. This maneuver can make the tip segment 39 and the bendable segment 30 looks like a “snack” movement. And operator can also rotate his or hers hand to align the tip distal curved region 38 with the vocal cords opening. Final goal is to move the tip distal curved region into the vocal cords opening.

### Third Embodiment

Same as previously, if an element is configured same with same function as in the first and second embodiment, same name and number label will be assigned. If an element has same function but configuration is not same as in the first and second embodiment, then a same name but a different number label will be assigned.

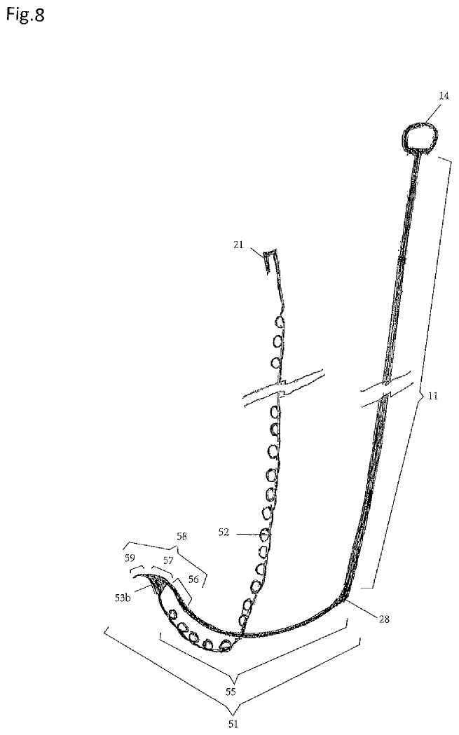

Third embodiment is based on the same concept and principle, but the third embodiment only comprises a rod, a retracting string without configuration of a second string. Referring to FIG. 8, showing a third embodiment, the device is in relaxed state before being inserted into an ET. The bendable member rod, includes a rod proximal segment 11 and a rod distal segment 51. The rod distal segment 51 can be further divided as a bendable segment 55 and a tip segment 58. The configuration of the rod ring 14, the rod proximal segment 11, the first bendable junction 28 between the rod proximal segment 11 and the rod distal segment 51 are completely same as in the first and second embodiments.

The string hook 21 has a same configuration as previous two embodiments. The retracting string will be configured same the coil-like or spring-like or wave-like extensible and compressible structure as in the first and second embodiment without being divided as a string proximal filament and a string distal filament, also called retracting string 52.

However the retracting string distal end spreads wider and then attaches the tip segment 58 from below, called the string distal attachment 53. The string distal attachment 53 can be configured into two or more forms. The string distal attachment 53 can be divided into three or more thinner filaments, called string attachment roots 53a, or is configured as a triangle-like plate with its base of the triangle like shape coupling with the tip segment 58, called sting attachment plate 53b. Preferably either the string attachment roots 52a or the string attachment plate 53b is coupled to lower surface of the tip middle section 57. Alternatively, the string attachment plate 53b can be configured as other geometric shapes, such as trapezoid, cubic, semi-circle, semi-ellipse and so on and the string distal attachment 53 can spread and attach anywhere of the tip segment 58 from below.

The shape, bendability, rigidity and materials made of the rod proximal segment 11 and the first bendable junction 28 are same as in the first and second embodiments. The rod proximal segment 11 will reduce the ET's original curvature. And same as in second embodiment, the bendable segment 55 is configured as a smooth bow curve shaped, but preferably shorter and deeper bow shaped. Preferably the bendable segment 55 is configured to have more rigidity. Therefore, in addition to its original curvature, the ET distal segment 43 will be bent more by the bendable segment 55. And at same time the curvature of the bendable segment 55 can be reduced by the curvature of the ET distal segment 43 in lesser extent when bendable segment is inside the ET. The bendable segment 55 has recoil or resilient property, once it comes out of the ET distal aperture, the bendable segment 55 will resume its original shape.

However the bendable segment 55 will still has more flexibility and bendability than the rod proximal segment 11.

The tip segment 58 is preferably configured as a crescent shape with its distal tip bending down and having its proximal end to end connection with distal end of the bendable segment 55. Or alternatively the tip segment 58 can be configured as a curved segment with distal portion of the tip segment bends down similar as in the first embodiment.

The tip segment 58 is divided into a tip distal section 59, a tip middle section 57, and a tip bendable section 56. Distal end of the bendable segment 55 is continuing and connecting to the tip bendable section 56 without a significant visible angle. The tip bendable section 56 is a smooth transition and responsible for majority of bending when the tip segment is pulled down by the retracting string 52, even though the whole tip segment 58 is flexible and bendable. Starting at distal end of the bendable segment 55, the cross section or diameter of the tip bendable section 56 become smaller and then keep same smaller diameter until beginning of the tip middle section, or slowly continue to become smaller until to the distal end of the tip distal section 59. The tip bendable section 56 is a short segment in length where the whole tip segment can be pulled from bellow by the retracting string 52. It is not a point. Alternatively, the tip bendable section 56 can be made with different materials as a “weak” segment therefore easily to be bent.

The tip distal segment 59 is to be advanced into vocal cords opening and guides the ET 40 into the vocal cords opening and trachea.

The string attachment roots 53a are three or more short and thin filaments at the distal end of the retracting string and spread out to couple to the tip middle section 57 from below. Its middle thin filament attaches the tip middle section with proximally 80-100 degree. When the retracting string 52 is pulled, multiple roots of the string attachment roots 53a will pull the tip middle section down therefore whole the tip segment 58 going down obliquely by bending the tip bendable section 56. Therefore the tip segment 58 can only be bent to down direction and to avoid the tip segment 58 being pushed to a too high position to lose alignment with the vocal cords opening.

Alternatively, the string distal attachment 53 can be configured as a single string directly attached to the tip middle section 57. Among all types of the string distal attachment 53, a triangle shaped the string attachment plate 53b is preferable. The base of triangle shaped string distal attachment 53b to attach to anywhere the tip segment, preferably to the tip middle section 57 and can bend the whole the tip segment down obliquely when is pulled by the retracting string 52.

At the set-up position, the retracting string 52 is more loosely and relaxed inside of the ET. At beginning of the curvature formation, distal sliding of the rod 10 makes the rod distal segment 51 curve up as the bendable segment 55 gradually comes out the ET distal aperture 48. Once the bendable segment 55 comes out of the distal opening of the ET 48, the bendable segment 55 will resume its totally original deeper curved bow shape which was inhibited by the ET when the bendable segment 55 was inside of the lumen of the ET. And once the bendable segment 55 comes out of the ET distal opening 48, the distal end of the bendable segment 55 will move up thereby moving whole the tip segment 58 into a higher position.

The further distal movement of the rod 11 will pull more of the retracting string outside of the ET distal aperture 48 and start to elongate the retracting string 52. Consequently elastic recoil force of the retracting string 52 will start to pull the tip segment 58. As the rod 10 is being pushed down more and retracting string 52 being stretched more, the tension on the retracting string 52 will increase, the retracting string 52 will exert more pulling force on the tip middle section 57 through the string distal attachment 53 therefore to bend the tip bendable section 56 and to pull down the tip segment 58 obliquely. After the bendable segment 55 has already sent the tip segment to a higher position, an obliquely down movement of the tip segment 58 and the up movement of the distal end of the bendable segment 55 will form a wave like or serpentine like curve. The tip segment 58 constitutes the down slope portion of the wave curve. Different directional movement of the bendable segment 55 and the tip segment 58 create the wave like bending curvature. Different forces to apply to the rod ring by an operator will generate different forms of the wave curves along the rod distal segment 51 therefore to form a “snake” like movement.

At the third stage of the curvature formation, a further pushing the rod ring will make the tip segment 58 being further pulled down obliquely which can make the deep bow shape become shallower in some extent and reverse some of up bending effect of the first bendable junction 28 in initial bending in some extent. Theses variables can be manipulated to create different design of the present invention to further make the rod distal segment 51 have more adaptability to accommodate a specific pharyngeal anatomy.

When the tip segment 58 meets a resistance or pressure from above, such as the anterior pharyngeal wall during advancing the rod 10, the tip segment 58 can be pressed down further and tip distal section 59 will point to more down direction. The surface of the tip segment 58 can be also very smooth and slippery either by well lubricating before its use or can be coated of polytetrafluoroethylene (PTFE) material or similar materials during the manufacture.

An operator's maneuver to generate the curvature is same as in the first and second embodiments. And the string attachment roots represents the string distal attachment 53 in the following drawings. But the string attachment roots can be replaced by the string attachment plate.

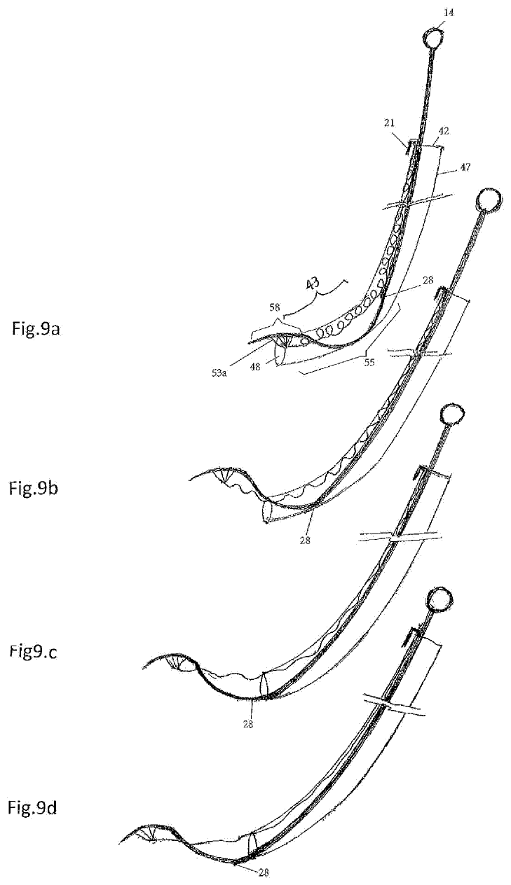

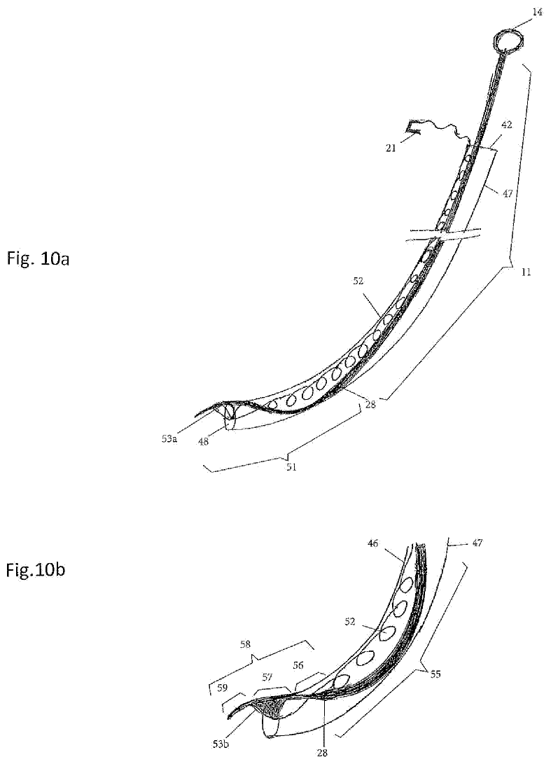

Referring to FIG. 9a, at a set-up position, the device has been inserted into an ET lumen and a portion of the rod distal segment 51 is positioned outside the ET distal aperture 48. An ET proximal end is held by an operator's hand same way as in the first embodiment and the second embodiments. The ET distal segment 43 is bent up more by the deep bow shaped curve of the bendable segment 55 because the bendable segment 55 has more rigidity than the ET distal segment 43. This is different in comparison with the first and second embodiments. The retracting string 52 is relaxed inside of the lumen of the ET.

Referring to FIG. 9b, the first push will slide more of the rod distal segment 51 out of the ET distal aperture 48 which will pull retracting string 52 in same direction. After the first push, the coiled-like or spring-like or wave-like extensible structure of the retracting string is taunted and is ready to be elongated. Because the bendable segment 55 is constructed with a preformed deep bow curve, the bendable segment 55 will gradually resume its original deep bow curve shape which was prevented by the ET while in the set-up position.

The first push is an early stage push, because the retracting string 52 is loosely fitted inside of tan ET, the tip segment 58 will not be pulled down significantly by the retracting string 52, instead that the tip segment 58 will be pushed up by the distal end of the bendable segment 55 as the bendable segment 55 moves more out of the ET distal aperture 48.

Referring to FIG. 9c, the second push will stretch the retracting string 52 and extend the string 52 about ⅓, and will slide the whole length of the bendable segment 55 just or near outside of the ET distal aperture 48. The retracting string 52 via the string attachment roots pulls the tip middle section, and the tip segment 58 will just slightly be pulled down. The first bendable junction 28 is near or just at the ET distal aperture 48.

Referring to FIG. 9d, during third push the rod 10 is pushed more distally. The retracting string 52 will stretch to ⅔ to ¾ of its total extensibility. The retracting string 52 will have more tension built on and will be more firmly holding the tip segment 58 downward obliquely. The first bendable junction 28 will be pushed more outside of the ET distal aperture 48. There are still some of residual extensibility left for the retracting, the operator can push more forward or pull back the rod ring 14 to reverse above curvature formation and therefore create a “snake” like movement along the rod distal segment. Even if the location of vocal cords can only be barely seen or can only be estimated, the operator can move the rod 10 toward that direction to “look for” the vocal cords opening. This maneuver is same as in the first and second embodiments. If there is a resistance or obstacle from above, the tip segment 58 can be further pressed or bent down.

Alternatively, all above three embodiments, the retracting string 52 can be designed to be pulled by an operator in addition to the rod 10 being pushed distally by the operator, or just by pulling the retracting string 20, 52 to pull the bendable segment back and generate curvatures.

What is claimed:

1. A stylet configured to be inserted inside of a tubular structure and to be pushed out of the tubular structure distal aperture comprising:
(a) a rod comprising a proximal segment and a distal segment, wherein the distal segment includes a bendable segment and a tip segment, wherein the proximal segment includes a proximal end and a distal end, wherein the bendable segment has a bendable segment proximal end and a bendable segment distal end, wherein the distal end of the proximal segment connects with the bendable segment proximal end and forms a first bendable junction, and wherein the bendable segment is configured to be pushed further out of the tubular structure distal aperture to form one or more curves along the bendable segment, wherein the bendable segment distal end connects with the tip segment and forms a second bendable junction, and wherein said second bendable junction is configured to be more flexible that the bendable segment;
(b) a retracting string, wherein the retracting string is configured as the coiled-like or spring-like or wave-like extensible and compressible structure which is extensible under an external pulling force, wherein the retracting string has a retracting string proximal end and a retracting string distal end; wherein the retracting string distal end is coupled to the bendable segment distal end or near bendable segment distal end, wherein at the retracting string proximal end is coupled to a docking device, wherein the docking device is configured to engage the of the retracting string proximal end to the edge of the proximal aperture of the tubular structure when the rod is moving distally, wherein the retracting string is configured to generate a retracting force when the retracting string is tauten and elongated therefore configured to hold the bendable segment backward by its natural recoil elasticity when the bendable member is being pushing forward, and thereby forming one or more curves along the bendable segment and moving the tip segment;
(c) a second string, wherein the second string has a second string first end and a second string second end, wherein the second string second end attaches to the bendable segment and the second string first end couples to the retracting string, and wherein the said second string first end divides the retracting string into a string proximal filament and a string distal filament.

2. The device of claim 1, wherein the proximal segment is configured to be stronger or stiffer than the distal segment and configured to withstand a pushing force exerted by an operator from its proximal end and transmit a pushing force to the bendable segment.

3. The device of claim 1, wherein the bendable segment is configured to be more flexible than the proximal segment, wherein the bendable segment further comprises a first bending section and a second bending section, wherein both the first and second bending sections are flexible and resilient, wherein the first bending section and second bending section are coupled thereby forming a third bendable junction, wherein the third bendable junction is configured with a preformed bent down angle of between 100-160 degrees.

4. The device of claim 1, wherein the docking device is a string hook, wherein the string hook is coupled to the proximal end of the retracting string and configured to hook to an anterior or lateral edge of a proximal opening of the tubular structure.

5. The device of claim 1, wherein the tip segment is configured to have a tip curved region in its long axis, wherein the second bendable junction is configured to be more flexible than the bendable segment, wherein the tip segment is made of flexible and resilient material and therefore able to bend if encountering pressure.

6. The device claim of 1, wherein the entire length or portion of length of the retracting string is configured as coiled-like or spring-like or wave-like extensible and compressible structure, wherein the coiled-like or spring-like or wave-like extensible and compressible structure is configured to have extensibility under a pulling force and same time its recoiled capability to generate a retracting force, wherein the retracting force or tension on the string proximal filament and the string distal filament is configured to change during the forming of the one or more curves.

7. The device of claim 6, wherein the string proximal filament and the string distal filament both are configured to extend longer and at the same time generate a pulling force to pull the bendable segment backward when the bendable segment is pushed forward to form a curve and before the second string participates the pulling process, wherein one of the functions of the string proximal filament is configured to pull the second string after the second string starts to become part of the pulling force, wherein the string proximal filament is configured to continuously increase tension as curvature formation progresses while the string distal filament is configured to not increase its tension after the second string participate in the pulling force.

8. The device claim 7, wherein the second string's second end is configured to couple to the second bending section just proximal to the third bendable junction, wherein after the second string starts pulling the second bending section the third bendable junction will carry less bending pressure therefore bend less or not to be bent.

9. The device claim 8, wherein the string distal filament of the retracting string is configured to not be fully extended after the second string has pull taut, therefore enabling the first bending section to be pressed down by using the third bendable junction as a pivot point.

10. The device of claim 2, wherein the first bendable junction is configured with sufficient rigidity to transmit the pushing force to the bendable segment distally without being significantly bent when it is still inside of the tubular structure, wherein the first bendable junction is configured to be bend when the it is pushed outside distal aperture of the tubular structure.

11. The device of claim 1, wherein second bendable junction is configured not to be directly bent by the retracting string pulling the bendable segment to form the curve along the bendable segment, wherein the second bendable junction is configured to be bent when the tip segment encounter a pressure from above.

12. A stylet configured to be inserted inside of a tubular structure and to be pushed out a tubular structure distal aperture to form one or more curves comprising:
(a) a rod, wherein the rod comprises a proximal segment and a distal segment, wherein the distal segment comprises a bendable segment and a tip segment, wherein the proximal segment has a proximal segment end and a proximal segment distal end, wherein the bendable segment has a bendable segment proximal end and a bendable segment distal end, wherein the proximal segment distal connects with the bendable segment proximal end and forms a first bendable junction, wherein the first bendable junction is configured to bend when a bending force is applied, and wherein said bendable junction being pushed outside of the tubular structure distal aperture is configured to be bend readily, wherein the tip segment having a tip segment proximal end and a tip distal curved region distally, wherein right or left side the tip segment is coupled to the bendable segment by a bendable side connection, wherein said bendable side connection is configured to be bend under a force;
(b) a retracting string, wherein the retracting string is configured as the coiled-like or spring-like or wave-like extensible and compressible structure which is extensible under an external force and has a retracting string proximal end and a retracting string distal end; wherein the retracting string distal end is coupled to the distal end or near the distal end of the bendable segment of the rod, wherein at the proximal end of the retracting string includes a string hook, wherein the string hook is configured to couple to the retracting string proximal end and configured to engage the proximal opening of the tubular structure when the rod is moved distally, wherein the retracting string is configured to generate a retracting force when the retracting string is tauten and elongated and therefore to hold the bendable segment of the rod backward when the rod is being pushing forward, and thereby forming one or more curves along the bendable segment of the rod;
(c) a second retracting string, wherein the second retracting string is a short segment string and has a second string's first end and a second string distal end, wherein the second string distal end couples to the tip segment proximal end, wherein the second string first end couples to the retracting string and divides the retracting string as string distal filament and a string proximal filament, therefore when the string proximal filament is being pulled the pulling force can be transmitted to the second string to pull the tip segment proximal end.

13. The device of claim 12, wherein the bendable segment is configured as a bow shaped curve and to be readily bent, wherein the bendable segment is flexible and resilient and can be bent into a deeper bow shaped when a bending force is applied.

14. The device of claim 12, wherein the tip segment of the rod is configured to have a tip distal curved region in its distal portion, wherein the tip segment is constructed with enough rigidity to move as a lever when the second string pulls up the tip segment proximal end.

15. The device of claim 14, wherein the bendable side connection between the bendable segment and the tip segment either on its right or left side is configured to bend and to twist when the tip segment proximal end is pulled up and the tip segment is moved as a lever.

16. The device of claim 15, wherein when the string proximal filament is configured to transmit a pulling force to the second string, consequently the second string will pull up the tip segment proximal end to cause the tip distal curved region tilting down when the rod is being pushed forward.

17. The stylet to be inserted inside of a tubular structure and to be pushed out a tubular structure distal aperture comprising:
(a) a rod comprising a proximal segment and a distal segment, wherein the distal segment includes a bendable segment and a tip segment, wherein the proximal segment includes a proximal end and a distal end, wherein the bendable segment has a bendable segment proximal end and a bendable segment distal end, wherein the distal end of the proximal segment connects with the bendable segment proximal end and forms a first bendable junction, wherein the first bendable junction is configured to be able to bend when a bending force applied, and wherein the bendable segment is configured to be pushed further out of the tubular structure distal aperture, and wherein said first bendable junction upon being pushed outside of the tubular structure distal aperture is configured to be bent readily, wherein the bendable segment distal end connects to the tip segment and forms a wave like shaped the distal segment of the rod;
(b) a retracting string configured as the coiled-like or spring-like or wave-like extensible and compressible structure which is extensible under an external force, wherein the retracting string has a retracting string proximal end and a retracting string distal end; wherein the retracting string distal end of the retracting string is configured to attach to the tip segment of the rod, wherein at the retracting string proximal end includes a docking device, wherein the docking device is configured in such way to couple the retracting string proximal end to the proximal aperture of the tubular structure when the bendable member is moving distally, wherein the retracting string is configured to generate a retracting force when the retracting string is tauten and elongated therefore pulls the tip segment down when the rod is pushing forward and thereby forming curvatures along the bendable segment of the rod.

18. The device of claim 17, wherein the bendable segment is made of material with enough rigidity to bend the tubular structure distal segment and same time be reduced the its curvature in some extent when the bendable segment is inside of the bendable segment, and wherein the bendable segment will resume its original curve when the bendable segment is outside of the tubular structure.

19. The device of claim 17, wherein the tip segment includes a tip bendable section, tip middle section and a tip distal section, therein the tip bendable section is connected with bendable segment of the bendable member and is configured to be bent when a force applied.

20. The device of claim 17, wherein the distal end of the retracting string can configured as either a string attachment root, a single string, or a string attachment plate, wherein the string attachment roots are is configured to divide into three more thinner filaments to attach along the bottom of the tip middle section, or wherein the string attachment plate is configured to attach the bottom of the tip middle section, wherein said single string is configured to attach the bottom of the tip middle section, and wherein when the retracting string is pulled the distal end of the retracting string is configured pull the whole the tip segment down.
